(19) United States
(12) Patent Application Publication
(10) Pub. No.: US 2013/0011392 A1
(43) Pub. Date: Jan. 10, 2013

(54) METHOD FOR ASSESSING THE ABILITY OF A PATIENT TO RESPOND TO OR BE SAFELY TREATED BY A NUCLEOSIDE ANALOG BASED-CHEMOTHERAPY

(75) Inventors: Joseph Ciccolini, Auriol (FR); Cédric Mercier, Marseille (FR); Jean-François Seitz, Marseille (FR); Laetitia Dahan, Marseille (FR); Athanassios Iliadis, Marseille (FR); L'Houcine Ouafik, Marseille (FR)

(73) Assignees: Assistance Publique Hopitaux De Marseille, Marseille Cedex 5 (FR); Universite D'Aix Marseille, Marseille Cedex 07 (FR)

(21) Appl. No.: 13/510,705

(22) PCT Filed: Nov. 19, 2010
(86) PCT No.: PCT/EP2010/067808

§ 371 Date: Sep. 7, 2012

(30) Foreign Application Priority Data
Nov. 20, 2009 (EP) 09306120.8

(51) Int. Cl. C12Q 1/34; A61P 35/00; A61K 33/24; A61K 31/7068; A61K 39/395

(52) U.S. Cl. 424/133.1; 435/18; 514/49; 424/141.1; 424/649

(57) ABSTRACT

The invention relates to an in vitro method for determining the ability of a patient with cancer to respond to a monochemotherapy or to a polychemotherapy involving the administration of at least one chemotherapeutic agent the liver elimination of which involves cytidine deaminase (CDA), or to be treated by at least one such chemotherapeutic agent, which method comprises determining the CDA activity in a biological sample of the patient, wherein a CDA activity above 6 U/mg of serum sample total protein is indicative of the inability of the patient to respond to the chemotherapy, a CDA activity between 1.1 U/mg and 6 U/mg of serum sample total protein is indicative of the ability of the patient to be treated by the chemotherapeutic agent when said agent is administered in the context of a monochemotherapy, and a CDA activity between 1.4 U/mg and 6 U/mg of serum sample total protein is indicative of the ability of the patient to be treated by the chemotherapeutic agent when said agent is administered in the context of a polychemotherapy.

The present invention relates to the field of cancer treatments. More particularly, it provides a method for determining the ability of a patient with cancer to respond to a monochemotherapy or to a polychemotherapy involving the administration of at least one chemotherapeutic agent the liver elimination of which involves cytidine deaminase (CDA), or the ability of a patient with cancer to be treated by at least one such chemotherapeutic agent.

### BACKGROUND

The gemcitabine is a nucleoside analog and an antimetabolite drug used in the treatment of various solid tumors in adult, including lung, pancreatic, or gynaecological cancers, and it can be considered as an attractive therapeutic option in children. Admittedly, 5-10% of severe toxicities are reported in patients treated with gemcitabine monochemotherapy, and 15-30% when the drug is given as a combination, i.e., in a polychemotherapy, (1, 2, 3).

Gemcitabine is characterized by a narrow therapeutic index with erratic pharmacokinetics, and its liver elimination depends upon a key enzymatic step, driven by cytidine deaminase (CDA). CDA is prone to genetic polymorphism, some mutations having been already reported as impacting on drug exposure-levels and related toxicities (1, 4). However, because genotype-to-phenotype relationships remain unclear with CDA, genotyping CDA as a marker for intolerance to gemcitabine could therefore be misleading (5, 6, 7, 8) and anticipating gemcitabine-induced toxicities solely on the basis of screening for genetic mutations can sometimes be uncertain, especially since epigenetic regulations of the CDA gene are still largely unknown. Inventors previously provided a method for assessing the risk of toxicity associated to a chemotherapeutic agent intake in a patient with cancer wherein metabolism of the chemotherapeutic agent involves CDA deaminase (WO2009/021551).

### SUMMARY OF THE INVENTION :

The inventors now advantageously propose determining the ability of a patient with cancer to respond to or be safely treated by a monochemotherapy or polychemotherapy implying the administration of at least one chemotherapeutic agent the liver elimination of which involves cytidine deaminase (CDA), such as gemcitabin, by phenotyping patients for their CDA activity prior to the prescription and administration of the chemotherapy.

For that purpose it is herein provided a simple, rapid and inexpensive method, based upon the assay of residual CDA activity in serum.

An object of the invention is thus an in vitro method for determining the ability of a patient with cancer to be treated by, i.e., to positively respond to, at least one chemotherapeutic agent the liver elimination of which involves cytidine deaminase (CDA), which method comprises determining the CDA activity in a biological sample of the patient, wherein:
(i) a CDA activity between 1,1 CDA activity unit per milligram of serum sample total protein (1,1 U/mg) and 6 CDA activity unit per milligram of serum sample total protein (6 U/mg) is indicative of the ability of the patient to be treated by the chemotherapeutic agent when said agent is administered in the context of a monochemotherapy,
(ii) a CDA activity between 1,4 CDA activity unit per milligram of serum sample total protein (1,4 U/mg) and 6 CDA activity unit per milligram of serum sample total protein (6 U/mg) is indicative of the ability of the patient to be treated by the chemotherapeutic agent when said agent is administered in the context of a polychemotherapy.

Another object is an in vitro method for determining the ability of a patient with cancer to respond to a monochemotherapy or to a polychemotherapy involving the administration of at least one chemotherapeutic agent the liver elimination of which involves cytidine deaminase (CDA), which method comprises determining the CDA activity in a biological sample of the patient, wherein a CDA activity below 6 U/mg is indicative of the ability of the patient to respond to the chemotherapy whereas a CDA activity above 6 U/mg is indicative of the inability of the patient to respond to the chemotherapy.

A monochemotherapy is a cancer treatment protocol implying the administration of only one chemotherapeutic agent. In the context of the present invention, the chemotherapeutic agent is an agent the liver elimination of which involves cytidine deaminase (CDA).

A polychemotherapy is a cancer treatment protocol implying the administration of several chemotherapeutic agents. In the context of the present invention, at least one of the chemotherapeutic agents used in the polychemotherapy is an agent the liver elimination of which involves cytidine deaminase (CDA). The other(s) chemotherapeutic agents may be any agent known and used in the context of a chemotherapy, such as, for example, an alkylating agent, an antimetabolite, an antimitotic agent, a monoclonal antibody, and a tyrosine-kinase inhibitor. The alkylating agent is preferably selected from oxaliplatin, cisplatin and carboplatin. The antimetabolite agent is preferably selected from capecitabine and 5-fluorouracil. Capecitabine (marketed as Xeloda®) is pentyl[1-(3,4-dihydroxy-5-methyl-tetrahydrofuran-2-yl)- 5-fluoro-2-oxo-1H-pyrimidin- 4-yl]aminomethanoate. The antimitotic agent is preferably selected from docetaxel and navelbin. The tyrosine-kinase inhibitor is preferably selected from erlotinib and sorafenib. The monoclonal antibody is preferably selected from trastuzumab (Herceptin®), bevacizumab (Avastin®) and cetuximab (Erbitux®).

In a particular embodiment, the chemotherapeutic agent the liver elimination of which involves cytidine deaminase (CDA) is gemcitabine. The chemotherapeutic agent may also be selected from Ara-Cytidine (Ara-C), CNDAC, decitabine, 5-aza-cytidine, clofarabine, nelarabine, troxacitabine, 2′-deoxy-2′-methylidenecytidine (DMDC) and tezacitabine the metabolism and liver detoxification of which are similar to those of gemcitabine.

The CDA activity may be determined by radioactive High Performance Liquid Chromatography (HPLC), or by spectrophotometry. The CDA activity is advantageously determined by visible spectrophotometry.

Another subject of the invention is a kit for determining the ability of a patient with cancer to be treated by at least one chemotherapeutic agent the liver elimination of which involves cytidine deaminase (CDA), such as gemcitabine, Ara-Cytidine (Ara-C), CNDAC, decitabine, 5-aza-cytidine, clofarabine, nelarabine, troxacitabine, 2′-deoxy-2′-methylidenecytidine (DMDC) and tezacitabine, which kit comprises:
i. a container comprising cytidine;
ii. a container comprising ammonium;
iii. a leaflet which describes a method for determining the cytidine deaminase (CDA) activity in a biological sample, by measuring the amount of ammonium released through conversion of cytidine into uridine by spectrophotometry, wherein the leaflet indicates that:
   a CDA activity between 1,1 and 6 U/mg, in a serum sample, is indicative of the ability of the patient to be treated by the chemotherapeutic agent the liver elimination of which involves CDA, when said agent is administered in the context of a monochemotherapy,
   a CDA activity between 1,4 and 6 U/mg, in a serum sample, is indicative of the ability of the patient to be treated by the chemotherapeutic agent the liver elimination of which involves CDA, when said agent is administered in the context of a polychemotherapy.

Also herein provided is a kit for determining the ability of a patient with cancer to respond to a monochemotherapy or to a polychemotherapy involving the administration of at least one chemotherapeutic agent the liver elimination of which involves cytidine deaminase (CDA) such as gemcitabine, Ara-Cytidine (Ara-C), CNDAC, decitabine, 5-aza-cytidine, clofarabine, nelarabine, troxacitabine, 2′-deoxy-2′-methylidenecytidine (DMDC) and tezacitabine, which kit comprises:
i. a container comprising cytidine;
ii. a container comprising ammonium;
iii. a leaflet which describes a method for determining the cytidine deaminase (CDA) activity in a biological sample, by measuring the amount of ammonium released through conversion of cytidine into uridine by spectrophotometry, wherein the leaflet indicates that a CDA activity below 6 U/mg, in a serum sample, is indicative of the ability of the patient to respond to the chemotherapeutic agent whereas a CDA activity above 6 U/mg is indicative of the inability of the patient to respond to the chemotherapy.

In an other embodiment, the present invention further provides a method for treating a patient with cancer, comprising, before the step of administering the chemotherapy to the patient, a step of determining the CDA activity in a biological sample of the patient in order to determine, using a method according to the present invention, whether a chemotherapeutic agent the liver elimination of which involves cytidine deaminase (CDA), is to be used or not in the chemotherapy for said patient.

### LEGENDS TO THE FIGURES

FIG. 1 shows the distribution of CDA activities in adult patients with or without early severe toxicities in subset 1 (monotherapy) and in subset 2 (polychemotherapy). A statistical difference was observed on CDA activities in the two subsets between patients with or without early severe toxicities upon gemcitabine intake (the lower the activity, the higher the risk to display toxicities). Cut-off associated with the toxic risk was 1.1 (gemcitabine—monotherapy) and 1.4 (gemcitabine combined with any other anticancer agent).

FIG. 2 shows High-resolution melting analysis of CDA single nucleotide polymorphisms 79A>C, 208G>A and 435C>T. Genomic DNA from reference patients were used for PCR and post-PCR melting curve analysis carried out on a LightCycler®480 instrument. Wild-type DNA was used as reference for each allele. The figure reveals that no correlation exists between mutations on the CDA gene (79A>C, 435T>C, 208G>A) and resulting enzymatic activities.

### DETAILED DESCRIPTION OF THE INVENTION :

The inventors have developed an in vitro method for determining the ability of a patient with cancer to respond to a monochemotherapy or to a polychemotherapy involving the administration of at least one chemotherapeutic agent the liver elimination of which involves cytidine deaminase (CDA), or to be treated by at least one such chemotherapeutic agent, which method comprises determining the CDA activity in a biological sample of the patient, wherein:
a CDA activity above 6 U/mg of serum sample total protein is indicative of the inability of the patient to respond to the chemotherapy,
a CDA activity between 1,1 U/mg and 6 U/mg of serum sample total protein is indicative of the ability of the patient to be treated by the chemotherapeutic agent when said agent is administered in the context of a monochemotherapy, and
a CDA activity between 1,4 U/mg and 6 U/mg of serum sample total protein is indicative of the ability of the patient to be treated by the chemotherapeutic agent when said agent is administered in the context of a polychemotherapy.

The present invention is based on a method of determining phenotypically CDA status in cancer patients, more particularly as an attempt to detect those who will be able to respond to a chemotherapeutic treatment and in particular those who will be treated by said treatment.

Inventors surprisingly discovered that a high CDA activity expressed in CDA activity unit per milligram of sample total protein (>6 U/mg) is an efficient marker to predict, in patients, the failure of a chemotherapeutic treatment implying the administration of at least one chemotherapeutic agent the liver elimination of which involves cytidine deaminase (CDA), such as gemcitabine. Usually, the therapeutic response is clinically evaluated 3 months after the beginning of the treatment. A treatment failure may be revealed by an increase of a tumoral marker (e.g. CA19-9 marker), and/or an increase of the tumor size, and/or an increase of the number and/or size of the metastases (evidenced by Pet-Scan).

A CDA activity, in a patient serum sample, superior to a control value of about 6U/mg (extensive CDA activity) is thus herein considered as abnormal and indicates the inability of the patient with cancer to respond to a monochemotherapy or polychemotherapy involving the administration of at least one chemotherapeutic agent the liver elimination of which involves CDA.

Inventors in addition identified specific CDA activity ranges usable as control ranges. The level of CDA activity in a serum sample of a given patient who is to receive chemotherapy has to be compared to said control ranges before any monochemotherapy or polychemotherapy, in order to select the appropriate treatment for said patient, i.e., a both non toxic and efficient treatment.

Inventors herein demonstrate that the CDA down-regulation can lead to toxic-death in patients exposed to a chemotherapeutic agent the liver elimination of which involves cytidine deaminase (CDA) whereas an abnormally increased (or extensive) CDA activity can neutralize the treatment and lead to the disease progression.

The control values of the ranges are cut-off values expressed in CDA activity unit per milligram of sample total protein (U/mg).

A CDA activity, in a serum sample, inferior to a control or cut-off value of about 1,1 U/mg is abnormal and indicative of a risk of severe toxicity upon gemcitabine uptake in the context of a monochemotherapy.

A CDA activity, in a serum sample, inferior to a control or cut-off value of about 1,4 U/mg is abnormal and indicative of a risk of severe toxicity upon gemcitabine uptake in the context of a polychemotherapy.

Gemcitabine (marketed as Gemzar®) is 2′-deoxy-2′,2′-difluorocytidine.

In another embodiment, the chemotherapeutic agent the liver elimination of which involves CDA is a nucleoside analog, e.g., an analog of purine or pyrimidine nucleosides. It is preferably a metabolite agent, preferably selected from the group consisting of cytarabine or Ara-Cytidine (Ara-C), CNDAC, decitabine, 5-aza-cytidine, clofarabine, nelarabine, troxacitabine, DMDC and tezacitabine.

Cytarabine (Ara-C) is 4-amino-1-beta-D-arabinofuranosyl-2(1H)-pyrimidinone.

CNDAC is 2′-C-cyano-2′-deoxy-1-beta-D-arabinofuranosylcytosine.

Decitabine is 5-aza-2′-deoxycytidine.

5-aza-cytidine is 4-amino-1-D-ribofuranosyl-s-triazin-2(1H)-one.

Clofarabine is 1,2,3,5-tetra-O-acetyl-β-D-ribofuranose.

Nelarabine is 2-amino-9-D-arabinofuranosyl-6-methoxy-9H-purine.

Troxacitabine is 4-amino-1-[(2S)-2-(hydroxymethyl)-1,3-dioxolan-4-yl]pyrimidin-2-one.

DMDC is 2′-deoxy-2′-methylidenecytidine.

Tezacitabine is (E)-2′-deoxy-2′-(fluoromethylene)cytidine.

Zalcitabine is dideoxycytidine.

A CDA activity, in a serum sample, inferior to a control or cut-off value of about 1,1 U/mg is abnormal and indicative of a risk of severe toxicity upon cytarabine or Ara-Cytidine (Ara-C), CNDAC, decitabine, 5-aza-cytidine, clofarabine, nelarabine, troxacitabine, DMDC and/or tezacitabine uptake, in the context of a monochemotherapy.

A CDA activity, in a serum sample, inferior to a control or cut-off value of about 1,4 U/mg is abnormal and indicative of a risk of severe toxicity upon cytarabine or Ara-Cytidine (Ara-C), CNDAC, decitabine, 5-aza-cytidine, clofarabine, nelarabine, troxacitabine, DMDC and/or tezacitabine uptake, in the context of a polychemotherapy.

Herein disclosed is therefore an in vitro method for determining the ability of a patient afflicted with a cancer to respond to a monochemotherapy involving the administration of at least one chemotherapeutic agent the liver elimination of which involves cytidine deaminase (CDA), which method comprises determining the CDA activity in a biological sample of the patient, wherein a CDA activity below 1.1 U/mg is indicative of a risk of severe toxicity due to the monochemotherapy.

Also herein disclosed is an in vitro method for determining the ability of a patient afflicted with a cancer to respond to a polychemotherapy involving the administration of at least one chemotherapeutic agent the liver elimination of which involves cytidine deaminase (CDA), which method comprises determining the CDA activity in a biological sample of the patient, wherein a CDA activity below 1.4 U/mg is indicative of a risk of severe toxicity due to the polychemotherapy.

In the context of the invention, the term <<early severe toxicity>>refers to a grade 3 toxicity or higher grade according to standard WHO grading (see Reporting Guidelines of the National Cancer Institute—http://ctep.cancer.gov/reporting/ctc.html) and occurring during or soon after the first or the second course of the protocol.

Haematological toxicities are leucopoenia, thrombopenia, anaemia, pancytopenia and putatively subsequent infectious diseases (such as fever, sepsis). Digestive toxicities are diarrhoea, mucitis, nausea or vomiting. Toxicity of the highest grade (grade 5) causes death of the patient.

Such a severe toxicity means that the chemotherapeutic agent is not suitable for the tested patient and may be life-threatening. Therefore, most preferably, the method of the invention should be performed in a patient before the chemotherapeutic agent is prescribed or administered in order to avoid extremely severe, and eventually lethal, toxicities after administration of a standard protocol implying the administration of at least one chemotherapeutic agent the liver elimination of which involves CDA.

The dosage regimen should then be adapted to the risk of severe toxicity herein determined or another chemotherapeutic agent which does not involve CDA for liver elimination, should be chosen instead.

In a preferred embodiment, the CDA activity is determined by spectrophotometry, preferably by visible spectrophotometry, most preferably at 630 nm.

More preferably, the CDA activity may be determined by measuring the amount of ammonium released through conversion of cytidine into uridine, by spectrophotometry.

The CDA activity is advantageously performed on a patient who does not exhibit an inflammatory syndrome (infection, sepsis, fever, tumor evolution, etc.) during or immediately before the sampling step.

In a particular method of the invention, the CDA activity is determined by:

a. incubating the biological sample with cytidine;

b. setting up a calibration curve of ammonium to be incubated similarly with the sample;

c. precipitating proteins so as to stop the reaction;

d. centrifuging and recovering the upper layer;

e. incubating the recovered upper layer of step d) with a mixture of phenol and sodium hypochlorite and recovering the upper layer comprising the ammonium;

f. detecting ammonium in the recovered upper layer of step e) with a spectrophotometer, preferably at 630 nm;

g. calculating the CDA activity, in regard with the signal/activity relationship generated by the calibration curve and the amount of proteins in the sample.

The protein amount of the sample is measured before incubating the sample with cytidine.

For each patient, one additional sample is incubated without the substrate (blank).

The biological sample may be a body fluid, such as serum, plasma, and blood. It may also be a tissue biopsy, in particular a liver biopsy.

Preferably, the biological sample is serum.

The patient is any human adult or child with cancer, for the treatment of which chemotherapy is needed. Solid tumors that may benefit gemcitabine, include lung, pancreatic, bladder, breast and gynaecological cancers. Cancers that may benefit from Ara-C, clofarabine, nelarabine and/or troxacitabine include acute myeloid leukemia, chronic myeloid leukemia, acute lymphoid leukemia, lymphomas. Cancers that may benefit from capecitabine include digestive cancers, in particular colorectal cancer, and breast cancer. Solid tumors (colorectal, lung) and haematological cancers may also benefit from CNDAC, decitabine, tezacitabine and/or DMDC. Cancers that can benefit from 5-aza-cytidine and/or decitabine are myelodisplastic syndromes.

In order to perform the method of the invention routinely, It is herein provided a kit for determining the ability of a patient with cancer to be treated by at least one chemotherapeutic agent the liver elimination of which involves cytidine deaminase (CDA) such as gemcitabine, Ara-Cytidine (Ara-C), CNDAC, decitabine, 5-aza-cytidine, clofarabine, nelarabine, troxacitabine, 2′-deoxy-2′-methylidenecytidine (DMDC) and tezacitabine, which kit comprises:
i. a container comprising cytidine;
ii. a container comprising ammonium;
iii. a leaflet which describes a method for determining the cytidine deaminase (CDA) activity in a biological sample, by measuring the amount of ammonium released through conversion of cytidine into uridine by spectrophotometry, wherein the leaflet indicates that:
   a CDA activity between 1,1 and 6 U/mg, in a serum sample, is indicative of the ability of the patient to be treated by the chemotherapeutic agent the liver elimination of which involves CDA, when said agent is administered in the context of a monochemotherapy,
   a CDA activity between 1,4 and 6 U/mg, in a serum sample, is indicative of the ability of the patient to be treated by the chemotherapeutic agent the liver elimination of which involves CDA, when said agent is administered in the context of a polychemotherapy.

Also herein provided is a kit for determining the ability of a patient with cancer to be respond to a monochemotherapy or to a polychemotherapy involving the administration of at least one chemotherapeutic agent the liver elimination of which involves cytidine deaminase (CDA) such as gemcitabine, Ara-Cytidine (Ara-C), CNDAC, decitabine, 5-aza-cytidine, clofarabine, nelarabine, troxacitabine, 2′-deoxy-2′-methylidenecytidine (DMDC) and tezacitabine, which kit comprises:
i. a container comprising cytidine;
ii. a container comprising ammonium;
iii. a leaflet which describes a method for determining the cytidine deaminase (CDA) activity in a biological sample, by measuring the amount of ammonium released through conversion of cytidine into uridine by spectrophotometry, wherein the leaflet indicates that a CDA activity below 6 U/mg, in a serum sample, is indicative of the ability of the patient to respond to the chemotherapeutic agent whereas a CDA activity above 6 U/mg is indicative of the inability of the patient to respond to the chemotherapy.

Preferably, the herein described kits are ready-to-use kits which, besides the leaflet, comprises seven containers:
a container comprising cytidine;
a container comprising ammonium;
a container comprising sodium and phosphate buffers;
a container comprising phenol and nitroprusside;
a container comprising tungstate;
a container comprising hypochlorite; and
a container comprising sulphuric acid.

In a preferred embodiment, the kit and the protocol may be as follows:
Buffers in the kit:
Solution A: KH2PO4 0.07M+Na2H2PO4, 0.07M PH7
Solution B: sodium tungstate (1g/10 ml H₂O)
Solution C: Ammonium (102 mg in 500 μL H₂O)
Solution D: Phenol (12 g phenol+60 mg sodium nitroprusside in 900 ml H₂O)
Solution E: Hypochlorite (1 g hypochlorite+8.4 g sodium hydroxyde in 1 l H₂O)
Solution F: cytidine 2 mM in solution A
Solution G: sulfuric acid 1 N.

Solutions are aliquoted as working solutions (vol: 1 ml, except for solution A: 20 ml and solution D: 15 ml) and stored at −80° C.
Protocol:

A blood sample is obtained when the patient is not undergoing chemotherapy and stored at 4° C. Preferably, the patient does not suffer from an inflammatory syndrome during or immediately before the sampling step.

The blood tube is centrifuged at 2500 rpm, during 20 min and at 4° C., then the serum fraction is isolated.

An aliquot of 50 μL is isolated and serum proteins are assayed according to a standard method, such as the Bradford method.

Three aliquots of 100 μL serum are isolated.

The ammonium control is prepared by mixing 10 μL solution C in 10 ml solution A=40 CDA Activity Units.

Dilution is performed (20/10/5/2.5 /1.25/0 A U.) in solution A.

The control and the sample may be incubated as follows according to a particular embodiment:

400 μl of solution F, 100 μL sample (n=2/patients), 100 μL sample (n=1/blank patient) or 400 μL of solution F+100 μL of each control curve point at 37° C. during 16 h.

The incubation is stopped by adding 400 μL of solution F into the blank samples. The reaction is stopped by precipitating proteins with 200 μl solution B+200 μL solution G.

After centrifugation (5000 rpm, room temperature,—5 min) 500 μL of supernatant are recovered.

Coupling is achieved by adding 1.5 ml solution D and 2 ml solution E.

The control and the sample may, according to another embodiment, be incubated as follows:

80 μl of solution F, 20 μL sample (n=2/patients), 20 μL sample (n=1/blank patient) or 80 μL of solution F+20 μL of each control curve point at 37° C. during 16 h.

The incubation is stopped by adding 80 μL of solution F into the blank samples. The reaction is stopped by precipitating proteins with 40 μl solution B+40 μL solution G. After centrifugation (5000 rpm, room temperature,—5 min) 25 μL of supernatant are recovered.

Coupling is achieved by adding 75 μl solution D and 100 μl solution E.

The mixture is then vortexed or shaken and incubated during 30 min at 37° C., before being read at 630 nm. The means of the two activities per patient is calculated after subtracting the blank value.

The CDA activity is expressed as Activity Units (U) taking into account the level of serum proteins.

The present invention further provides a method for treating a patient with cancer, comprising, before the step of administering the chemotherapy to the patient, a step of determining the CDA activity in a biological sample of the patient in order to determine, using a method according to the present invention, whether a chemotherapeutic agent the liver elimination of which involves cytidine deaminase (CDA), is to be used or not in the chemotherapy for said patient.

The below examples illustrate the invention without limiting its scope.

### EXAMPLES

### Example 1

### Deregulation in Cytidine Deaminase (CDA) is Associated with Increased Risk of Developing Early Severe Toxicities Upon Drug Exposure

The main objective was to evaluate the relevance of CDA testing, both on a genotype and on a phenotype-basis, as a means to identify patients who developed early severe toxicities upon gemcitabine intake. To this end, inventors validated in an animal model a simple, rapid and cheap phenotypic method to determine CDA activity. Next, they studied the relevance of this CDA testing as a marker for early severe toxicities in 64 adult patients undergoing gemcitabine-alone treatment (monochemotherapy). Then, robustness of this test was further checked by adding 66 extra-patients treated with combinational therapies (polychemotherapy) to reach a final subset of 130 adult patients, and eventually by performing the test in 20 heavily pre-treated children receiving gemcitabine. Additionally, screening for the most usual mutations reported on the CDA gene (e.g., 79A>C, 208G>A and 435C>T polymorphisms) was carried out in adults.

Patients and Methods

Animals:

Experiments were carried out in 4-week old male swiss mice (Janvier, Le Genest St Isle, France). CDA deficiency was achieved by treating half animals with tetrahydrouridine (THU) (100 mg/kg) as described previously (9). Animal care was in agreement with animal welfare guidelines of our institution.

Animal Pharmacokinetics and Toxicity Monitoring:

Drug pharmacokinetics and haematological toxicity monitoring was evaluated in 24 mice injected intraperitoneally with gemcitabine (100 mg/kg), 12 of them having been previously administered with THU. Parent-drug and difluorodeoxyuridine (dFdU) metabolite concentrations in plasma were assayed by UV-HPLC, as described in the literature (10). Elimination rate of gemcitabine was evaluated through a compartmental analysis and a stochastic model, using a home-built algorithm (11). Mean metabolization ratio for gemcitabine was calculated as follows: dFdU (μg/ml)/gemcitabine (μg/ml)×100. Carcass weights were monitored. Drug-induced leucopenia was the marker for gemcitabine-related toxicity. Leucocytes count was performed on day-0, then day-2, day-5 and day-8 post injection using Flow Cytometry analysis.

CDA Testing in Animals:

CDA status was evaluated by measuring residual enzymatic activity in serum, as described previously (12). Briefly, cytidine conversion to ammonium was monitored spectrophotometrically after overnight incubation at 37° C. CDA activity was expressed as U/mg proteins, with 1U=4.10⁻³ μMol of ammonium released per minute and per milliliter of serum. Proteins were assayed using the standard Bradford method. Each sample was assayed in triplicate.

Patients:

A total of 150 cancer patients (130 adults—Table 1 and 20 children—Table 2) hospitalized at La Timone University Hospital of Marseille, France, from January 2007 to December 2008, were enrolled in this retrospective, observational study. Written informed consent and local ethic committee approval were obtained prior to performing CDA testing. Patients were divided into 3 subsets:

TABLE 1
Adult Patients treated with gemcitabine alone or in combination:
demographics and clinical characteristics.

| | n | % |
|---|---|---|
| Monotherapy | | |
| Patients | 64 | 100 |
| Age (years) | | |

| | |
|---|---|
| Median | 63 |
| Range | 40-84 |

| | | |
|---|---|---|
| Female sex | 30 | 47 |
| Primary tumor location | | |
| Pancreatic | 40 | 62 |
| Gynaecological | 7 | 11 |
| Lung | 3 | 5 |
| Vesical | 3 | 5 |
| Liver | 2 | 3 |
| Other | 11 | 14 |
| Site of metastases: | | |
| Liver | 24 | 38 |
| Lung | 11 | 17 |
| Peritoneal carcinosis | 6 | 9 |
| Other/multiple | 13 | 20 |
| No metastase/unknown | 10 | 16 |
| Gemcitabine dosage (mg/m²) | | |

| | |
|---|---|
| Median/Mean | 1000/984 |
| Range | 500-1250 |
| Polytherapy | |

| | | |
|---|---|---|
| Patients | 66 | 100 |
| Age (years) | | |

| | |
|---|---|
| Median | 61 |
| Range | 42-80 |

| | | |
|---|---|---|
| Female sex | 26 | 39 |
| Primary tumor location | | |
| Vesical/urothelial | 20 | 30 |
| Breast | 13 | 19 |
| Gynaecological | 12 | 18 |
| Pancreatic | 12 | 18 |
| Other | 9 | 14 |
| Site of metastases | | |
| Lung | 32 | 48 |
| Liver | 27 | 41 |
| Bone | 20 | 30 |
| Other/unknown | 23 | 35 |
| Concomitant treatment | | |
| CBDCA/CDDP | 19 | 29 |
| Docetaxel | 17 | 28 |
| Oxaliplatin | 8 | 12 |
| Other | 22 | 33 |
| Gemcitabine dosage (mg/m²) | | |

| | |
|---|---|
| Median/Mean | 1000/1038 |
| Range | 600-1250 |

Subset 1: 64 patients (34 men/30 women, mean and standard deviation [SD] age, 63±14 years) treated with gemcitabine (mean dose: 984±158 mg/m²). These patients were mostly treated for pancreatic (n=40 patients) or gynaecological (n=7 patients) cancers. Other tumour types included liver, lung, vesical, or other localizations. In patients with digestive cancers, gemcitabine was administered as a fixed-dose rate infusion, whereas other patients received a standard rapid infusion.

Subset 2: 66 extra-patients (26 women/40 men; and SD age, 61±14 years), all being treated with gemcitabine associated with various anticancer drugs, were added to the previous population to reach a final, heterogeneous subset of 130 adult patients. In the 66 extra-patients, gemcitabine (mean dose: 1038±177 mg/m²) was associated mainly with CDDP/CBDCA (n=29 patients), with docetaxel (n=17 patients) and with oxaliplatin (n=8 patients). Remaining patients were given various drug combinations including capecitabine, fluorouracil (5-FU), herceptin, navelbine, sorafenib, axatinib, and erlotinib. Gemcitabine was given either as a rapid infusion or at fixed-dose rate infusion, depending on tumor location, as described previously. Twenty patients were treated for vesical-urothelial cancers. Other tumour types were pancreatic (n=12 patients), breast (n=12 patients), gynaecological (n=12 patients), and other, including lung, head and neck, liver, and other localisations.

Subset 3: 20 patients (12 men/8 women; mean and SD age, 12±7 years) from the Pediatric Oncology Unit of La Timone University Hospital that had been previously treated with gemcitabine-based protocols had their CDA status phenotyped as well (Table 2). Most of these young patients were heavily pre-treated, and gemcitabine was mostly given as part of a Gemox protocol (n=14 patients), or associated with paclitaxel (n=3 patients) or with various other anticancer drugs (e.g.

carboplatine, liposomal doxorubicin) or dexamethasone. In children, mean gemcitabine dose was 1028±190 mg/m² (minimum, 600 mg/m² —maximum, 1400 mg/m²). Tumour types were extremely heterogeneous and included osteosarcoma, medulloblastoma, Ewing's sarcoma, and Hodgkin's syndrome.

TABLE 2
Paediatric Patients: demographics and clinical characteristics

| | |
|---|---|
| Patients | 20 |
| Age (years) | |
| Median | 12 |
| Range | 4-27% |

| | n | % |
|---|---|---|
| Female sex | 8 | 40 |
| Primary tumor location | | |
| Osteosarcoma | 2 | 10 |
| Ewing's sarcoma | 1 | 5 |
| Hodgkin disease | 1 | 5 |
| Medulloblastoma | 1 | 5 |
| Rare tumours | 15 | 75 |
| Concomitant treatment | | |
| Oxaliplatin | 14 | 70 |
| Docetaxel | 3 | 15 |
| Other | 3 | 15 |
| Gemcitabine dosage (mg/m²) | | |

| | |
|---|---|
| Median/Mean | 1000/1027 |
| Range | 600-1400 |

Sampling:

All patients had a single, 3-ml blood sample withdrawn out of the gemcitabine infusion. Tubes were kept at 4° C. up to 6 h maximum until centrifugation at 2500 rpm for 20 minutes. Serum fraction and cell pellet containing DNA were then stored at −80° C. until they were analyzed.

Phenotypic CDA Status Determination:

CDA status was evaluated by measuring residual enzymatic activity in serum, as described previously (12) and above.

Toxicity Monitoring:

Toxicities were graded following the standard National Cancer Institute-Common Toxicity Criteria guidelines. Toxicities of grade ≧3 were regarded as “severe”. A toxic event was considered “early” when occurring after the 1^{st} or the 2nd day of the gemcitabine course, and was considered “late” when occurring after those days.

CDA Genetic Mutations Screening:

The presence of the 79A>C, 218 G>A and 435 T>C polymorphisms of the CDA gene was verified after DNA isolation from whole-blood cells in adult patients, following a standard extraction procedure. The assay was based on different melting temperatures (Tm) of fluorescent-labeled oligonucleotide hybridization probes, using a single-step assay that combines fluorescence Polymerase Chain Reaction (PCR) with melting curve analysis, as previously described (12). Analyses were performed using the Light-Cycler 480 technology (Roche Diagnostics, France).

Statistical Analysis:

Statistical significance was tested using Sigma Stat software (Jandel Scientific, Germany) with appropriate testing. Student or Mann-Whitney Rank sum tests were performed, according to data distribution. A p value of 0.05 was regarded as statistically significant. Cut-off in CDA values associated with the event “early severe toxicity” was calculated using the Discrimination Function of Matlab 7.0 software (1994-2009 The MathWorks, Inc.).

Cut-off in CDA values associated with the occurrence “Severe early toxicities upon gemcitabine intake” were determined using a biostatistical method based upon the Classreftree function of Mathlab software. The event “Early severe toxicity/No early severe toxicity” was set as the categorical variate of the algorithm. Determination of the output of this covariate according to a predictive function (“CDA activity in serum”) was performed for two subsets of patients: subset “monotherapy” (64 patients) and subset “polychemotherapy” (66 patients).

Results

CDA Deficiency in Mice

CDA activity was competitively inhibited by injecting THU prior to gemcitabine administration. CDA functional testing was performed, using serum residual activity as a surrogate marker for overall CDA status in animals. Mean CDA activity recorded in swiss mice was 7.7 U/mg. In animals pre-treated with THU, CDA activity fell down by 87% (0.98 U/mg, p<0.01, Student).

Animal Pharmacokinetics Studies

Gemcitabine exposure in animals, as estimated with calculation of the area under curves (AUC₀₋₆₀) was found to be 2.6-fold higher in animals with inhibited CDA (e.g., 142 vs. 54 μg/ml/min) (Figure not shown). Normal gemcitabine clearance in mice was 1.26.10⁻² L/min/kg. In animals with altered CDA function, gemcitabine clearance decreased by 74%, (3.25.10⁻³ L/min/kg). Accordingly, drug monitoring of dFdU showed lower levels of circulating metabolite in plasma. Mean metabolization ratio was 28±5.9 in mice with functional CDA, and decreased to 3±2 in mice with inhibited CDA (Figure not shown).

Animal Toxicities

Leucocytes count was monitored over a 8-day period after gemcitabine injection. In mice with normal CDA activity, mild leucopenia was observed on day-5 (mean leucocytes count: 3.2 G/L, 23% decrease as compared with untreated animals) and all animals spontaneously recovered from their toxicities by day 8. In CDA-deficient animals treated with gemcitabine, a sharper decrease (1.5 G/L, 65%) was observed on day 5 and all animals were dead by day 8. Toxic-death was so quick that no difference in carcass weights was evidenced (data not shown).

Treatment-Related Toxicities in Adults.

Subset 1: 7 of 64 patients (11%) experienced early severe toxicities after administration of gemcitabine alone. Table 3 shows the repartition in toxicity type and schedule of occurrence in patients treated with gemcitabine monotherapy.

TABLE 3
Treatment-related toxicities according to NCI-CTC criteria
(Subset1, n = 64).

| | Early | Late |
|---|---|---|
| Toxicities grade 3/4 | n (%) | n (%) |
| Haematological Toxicities | | |
| Leucopenia | 7 (11%) | 10 (16%) |
| Neutropenia | 6 (9.6%) | 8 (12.8%) |
| Anemia | 1 (1.6%) | 1 (1.6%) |
| Thrombopenia | 2 (3.2%) | 2 (3.2%) |
| Sepsis | 3 (4.8%) | 0 (0%) |
| Non-Haematological | | |
| Toxicities | | |
| Asthenia | 1 (1.6%) | 1 (1.6%) |
| Diarrhea | 0 (0%) | 1 (1.6%) |

Subset 2: 9 of 66 of the extra-patients added (14%) had severe side-effects after being treated with gemcitabine in combination with another anticancer drug. Overall, in this subset, 16 adult patients of 130 (12%) experienced early severe toxicities after the first or second administration of a gemcitabine-containing protocol. Most frequent early severe toxicities encountered were haematologic (i.e., leucopenia, neutropenia, thrombocytopenia) and asthenia. Most frequent late or mild toxicities were haematologic, vomiting, diarrhea, and anorexia. There was no statistical difference in gemcitabine dosage between patients with and without early severe toxicities (952 vs 984 mg/m²). Similarly, no difference in age (64 vs 62 years) or in gender repartition was observed between the two groups.

Treatment-Related Toxicities in Children:

No early severe toxicities were observed in subset 3. Late/cumulative toxicities included grade-3 neutropenia (n=4 patients, 20%), anemia (n=4 patients, 20%) and thrombopenia (n=2 patients, 10%).

CDA Activity in Patients:

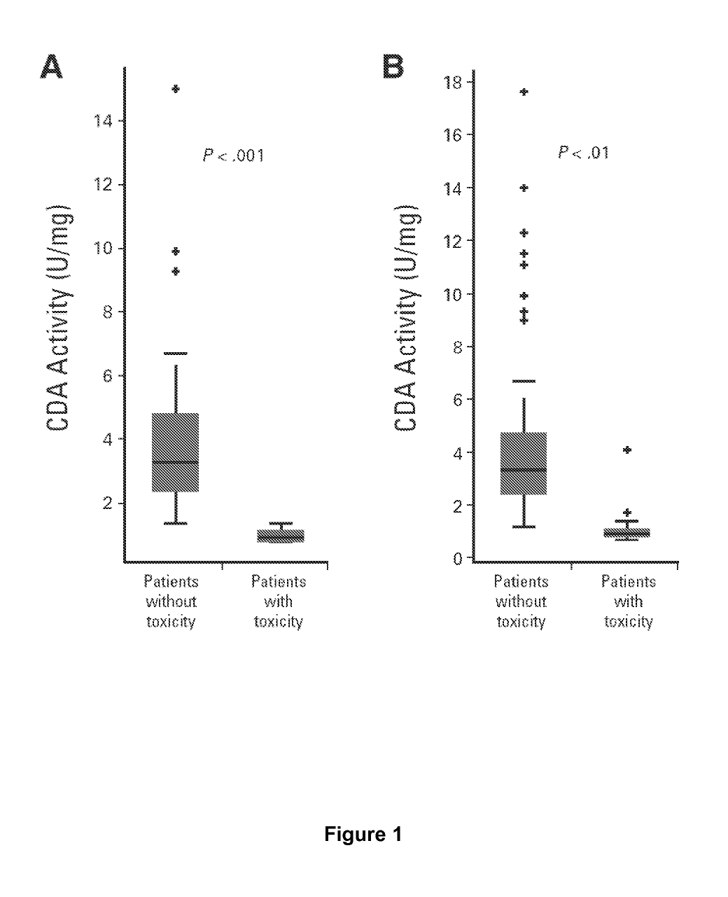

When undergoing gemcitabine-only treatment (monochemotherapy), CDA activity in patients without toxicities was 3.9±2.4 U/mg. In patients having experienced early severe toxicities, CDA was 1±0.2 U/mg (p<0.001, Mann-Whitney Rank sum test, FIG. 1A). The cut-off statistically associated with the event of “early severe toxicity” was 1.40 U/mg in this subset. Presence of liver metastases was not associated with diminished CDA activity (data not shown).

When adding the 66 additional patients treated with combinational therapy (polychemotherapy), average and SD CDA activity in adults was 3.6+/−2.7 U/mg (minimum 0.65 U/mg; maximum: 17.6 U/mg; n=130). Mean and SD CDA activity in patients without early toxicities was 4±2.6 U/mg (min 1.2-max 17.6) and mean and SD CDA activity in patients with early toxicities after gemcitabine intake was 1.2±0.8 U/mg (minimum, 0.65 U/mg—maximum 4.1 U/mg). The two groups were significantly different (p<0.01, Mann-Whitney Rank sum test—FIG. 1B), and the cut-off associated with the event of “early severe toxicity” was 1.1 U/mg. Of note, 9 of 130 adult patients (7%) displayed markedly low CDA activity (e.g., <1 U/mg), and all of them had >grade-3 toxicities, 4 of them having been treated with gemcitabine alone. In children, mean and SD CDA activity was 3.5+/−2.6 U/mg. and none of the 20 young patients had CDA activity <1 U/mg. There was no statistical difference between adult and children CDA values (p>0.05, Mann-Whitney Rank sum test).

CDA Mutations Screening in Patients:

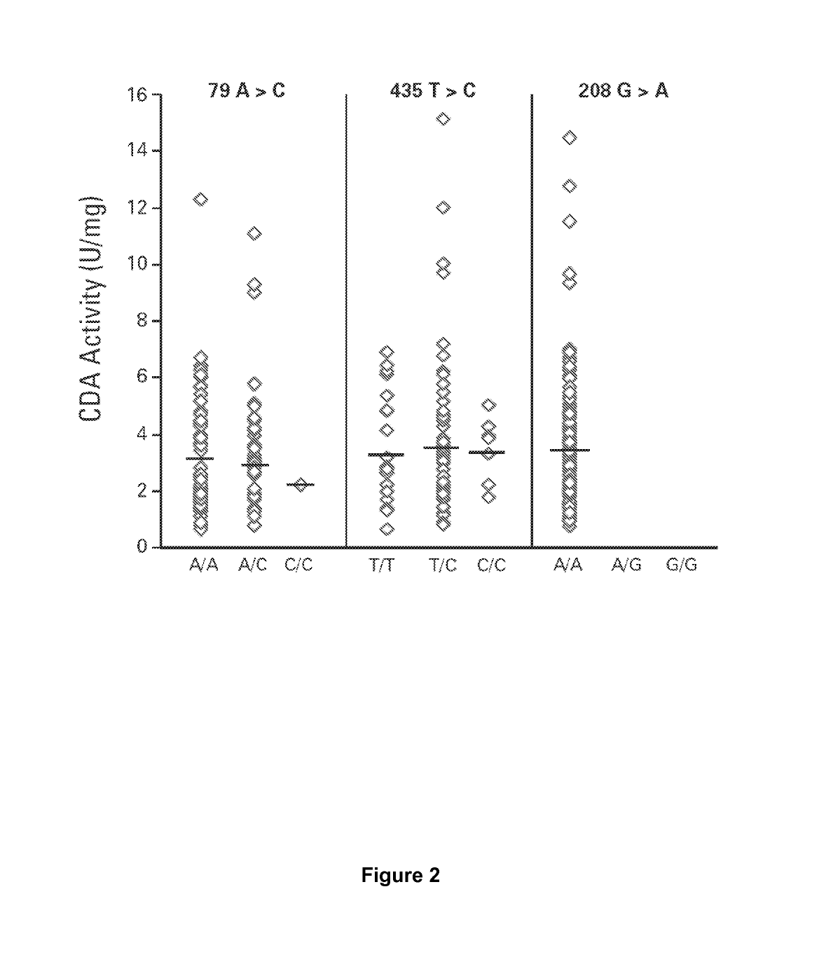

Genetic screening was performed in 109 adult patients (21 samples failed in yielding sufficient DNA for subsequent analysis). No 208G>A mutation was found. Heterozygoty on the 79A>C mutation was observed in 47% of the patients (mean and SD CDA activity: 3.5±1.7 U/mg), 1.1% were homozygous (mean CDA activity: 2.2 U/mg) and the 51% remaining ones were WT (mean CDA activity: 3.6±1.7 U/mg). 68% of the patients were heterozygous for the 435T>C mutation (mean and SD CDA activity: 3.6±1.8 U/mg), 10% were homozygous (mean and SD CDA activity: 3.5±1.8 U/mg) whereas 22% were WT (mean and SD CDA activity: 3.3±1.8 U/mg). No relationship was found between CDA genotypic status on the 79A>C and 435T>C mutations and resulting activities (FIG. 2). Consequently, no relationship was found between CDA genotypic status on the 79A>C and 435T>C mutations and toxicities. A number of individuals had multiple mutational status, with no impact on CDA levels either (data not shown).

Results:

Inventors voluntarily included totally unselected and heterogeneous patients, either from adult or paediatric oncology units, so as to evaluate the reliability of the test in a daily clinical practice.

Animal data first confirmed the major impact CDA function had on gemcitabine pharmacokinetics profile, since a near 3-time increase in drug exposure with subsequent lethal toxicities was evidenced in animals with CDA deficiency. Importantly, measuring residual CDA in serum fully allowed inventors to discriminate animals with severe toxicities from those without severe toxicities, thus demonstrating that their test was fully predictive for toxicities.

In human, 12% of adult patients experienced early severe toxicities after gemcitabine administration. A significant difference in CDA activities was observed between patients with and without toxicities (1.2±0.8 U/mg vs 4±2.6, p<0.01). Conversely, no genotype-to-phenotype relationships were found. Of note, the patients displaying particularly reduced CDA activity all experienced strong toxicities. Gemcitabine was well tolerated in children and no CDA deficiency was evidenced.

Discussion:

In adults, a wide inter-patient variability in CDA values was recorded (min 0.65, max 17.6 U/mg), an observation consistent with the erratic pharmacokinetics profile of gemcitabine abundantly described in the literature (21, 22). Inventors first evaluated the relevance of CDA testing in an adult subset limited to patients treated with gemcitabine monotherapy only. Because they evidenced that average CDA level in patients with toxicities was 3.9-fold lower than in patients without toxicities, it is much likely that impairment in drug detoxification was a culprit for the early severe toxicities observed. Besides, the highly significant difference in CDA activities recorded in toxic and non-toxic patients demonstrates that CDA functional testing is a predictive marker for toxicities allowing the detection of patients with reduced activities before any chemotherapeutic treatment.

Interestingly, after adding 66 extra-patients treated with a wide variety of drug combinations, inventors found that reduced CDA activity was still associated with toxicities. This observation suggests that CDA testing could be of interest in any adult patients treated with gemcitabine, either administrated alone or concomitantly with other cytotoxic drugs. Importantly, they observed that a CDA value of 1,1 U/mg in monochemotherapy, and a CDA value of 1,4 in polychemotherapy, was respectively the limit below which the risk of developing early severe toxicities with gemcitabine was maximal, because 100% of these patients experienced severe toxicities. In this study, 7% of adult patients displayed such markedly low CDA activities, an observation in line with the 5-10% incidence of early severe toxicities reported in the literature with gemcitabine.

Additionally, inventors studied CDA activities in children having been treated with gemcitabine. Despite disappointing studies when used alone, gemcitabine in combination with either vinorelbine or taxanes has recently shown efficiency in children with refractory Hodgkin disease or bone sarcoma (23, 24). In this respect, an increasingly growing number of young patients likely to receive gemcitabine are expected, and having a marker for tolerance in such usually heavily pre-treated population would be of interest. Mean CDA activities in children were comparable to the distribution observed in adult patients. In the context of the present invention, no young patients could be considered as CDA-deficient (e.g., with activity <1,1 U/mg) , and none of them presented early severe toxicities, despite the fact that up to 1400 mg/m² of gemcitabine was given. This confirms that the cut-off is similar to that of adult.

Finally, genotypic investigations were also carried-out in adults on the 208G>A, 435C>T and 79A>G polymorphisms, and were not conclusive. No patients bearing the 208G>A mutation was found; an observation consistent with the fact that this polymorphism has been only described in Japanese patients so far (4, 14, 15). More surprisingly, no genotype-to-phenotype relationship was evidenced with the 79A>C polymorphism, a finding contradictory to the data recently published by Tibaldi et al. and Giovanetti et al. (1, 8). This discrepancy is surprising, especially because the incidences of the 79A>C mutation were fully comparable between the studies. Unaddressed epigenetic deregulations may explain this difference, because the whole spectrum of upstream events potentially affecting CDA functionality is far from being all elucidated (16). Besides, contradictory conclusions have already arisen in the literature from the study of the functional impact of the 79A>C mutation (6, 7, 8). In this respect, addressing CDA status issue primarily through a phenotypic approach, rather than genotyping, appears to be an appropriate option to detect patients at risk. In our study, despite the huge heterogeneity in patients characteristics, CDA functional testing proved to be a reliable marker for detecting adult patients displaying early severe toxicities after gemcitabine-containing therapy.

Conclusion:

CDA functional testing is a simple and easy marker to discriminate patients at risk of developing severe toxicities with gemcitabine. Concretely, patients with cancer can be considered as CDA-deficient, and are likely to experience early severe toxicities in the context of a monochemotherapy with gemcitabine, when their CDA activity is below 1,1 U/mg, and in the context of a polychemotherapy implying the administration of gemcitabine, when their CDA activity is below 1,4 U/mg.

### Example 2

### Extensive CDA Activity is Related to Treatment Failure in Digestive Cancer Patients Treated with Gemcitabine-Based Chemotherapy

Gemcitabine is a mainstay in the treatment of billiary and pancreatic cancers, either alone or associated with another chemotherapy, such as oxaliplatin or a target therapy implying the use of a monoclonal antibody (for example herceptin) or of a tyrosine-kinase inhibitor (for example erlotinib or sorafenib).

As explained previously a reduced CDA activity is a marker for early severe toxicities upon gemcitabine intake. Inventors further observed that about 10% of patients displayed particularly elevated CDA activities. They discovered that patients with high CDA activities experienced more frequently treatment failure after being administered with standard dosage of gemcitabine than patients with normal CDA activities when compared to standard values.

Patients and Methods:

A total of 40 adult patients (21 men/19 women; 61±11 years old) with mostly pancreatic cancers (n=36 patients) and other digestive localizations (n=4 patients) were included in this retrospective study. All patients had been treated with gemcitabine alone (n=23 patients), associated with oxaliplatin (n=7 patients), erlotinib (n=2 patients), capecitabine (n=1 patient) or other drugs, including molecules used in target therapies (n=7 patients). Mean gemcitabine dose was 980 mg/m² (minimum: 500 mg/m², maximum: 1250 mg/m²). CDA activity was measured spectrophotometrically from serum samples. Search for three genetic mutations (79A>C, 435T>C and 208G>A) was additionally performed. The cut-off associated with CDA extensiveness (extensive CDA activity) was set at 6 U/mg (e.g., >25% of the mean value of the population). Treatment efficacy was evaluated 3 month after beginning of the chemotherapy by CT imaging, with or without additional monitoring of the CA19-9 tumor marker.

Results:

CDA activities recorded in the whole population ranged from 0.8 to 17.4 U/mg (mean=4.1 U/mg, median=3.4 U/mg). No correlation between genotype and phenotype was evidenced. 8 out of 40 patients (20%) displayed CDA activities associated with an extensive phenotype (minimum: 6 U/mg—maximum: 17.4 U/mg). These extensive patients (4 men/4 women; 58±10 years old; mean gemcitabine dosage: 960 mg/m²) did not differ from the non-extensive ones (tumor localization, treatment type, clinical covariates). A significant difference was evidenced in control disease between extensive (CDA>6 U/mg, n=8) and non-extensive patients (CDA<6 U/mg, n=32). In the extensive subset, 2 patients (25%) had controlled disease (1 response, 1 stable disease) and 6 were non-responder with progressive disease (75%) whereas in the non-extensive subset, 27 patients (84.5%) had controlled disease and 5 (15;5%) has progressive disease (p<0.001). Median CDA activity was 2.8 U/mg in patients with controlled disease [1.6 U/mg—3.8 U/mg] whereas median CDA activity was 7.95 U/mg [3.8 U/mg—17.4 U/mg] in non-responders (p<0.001).

Conclusion:

Despite the heterogeneity of the patients and treatment type of this pilot, retrospective study, our clinical data provide a clear demonstration that high CDA activity (>6 U/mg) is an efficient marker for treatment failure in patients with gemcitabine-containing chemotherapy.

References

1. Tibaldi C, Giovannetti E, Vasile E, et al. Correlation of CDA, ERCC1, and XPD polymorphisms with response and survival in gemcitabine/cisplatin-treated advanced non-small cell lung cancer patients. Clin Cancer Res. 14: 1797-803, 2008.

2. Tanaka T, Ikeda M, Okusaka T, et al. Prognostic factors in Japanese patients with advanced pancreatic cancer treated with single-agent gemcitabine as first-line therapy. Jpn J Clin Oncol. 38: 755-61, 2008.

3. Lee J O, Kim D Y, Lim J H, Seo M D, et al. Palliative chemotherapy for patients with recurrent hepatocellular carcinoma after liver transplantation. J Gastroenterol Hepatol. [Epub ahead of print] 2008.

4. Yonemori K, Ueno H, Okusaka T, et al. Severe drug toxicity associated with a single-nucleotide polymorphism of the cytidine deaminase gene in a Japanese cancer patient treated with gemcitabine plus cisplatin. Clin Cancer Res. 11: 2620-4, 2005.

5. Kirch H C, Schroder J, Hoppe H, et al. Recombinant gene products of two natural variants of the human cytidine deaminase gene confer different deamination rates of cytarabine in vitro. Exp Hematol. 26: 421-5, 1998.

6. Yue L, Saikawa Y, Ota K, et al. A functional single-nucleotide polymorphism in the human cytidine deaminase gene contributing to ara-C sensitivity. Pharmacogenetics. 13: 29-38, 2003.

7. Gilbert J A, Salavaggione O E, Ji Y, et al. Gemcitabine pharmacogenomics: cytidine deaminase and deoxycytidylate deaminase gene resequencing and functional genomics. Clin Cancer Res. 12: 1794-803, 2006.

8. Giovannetti E, Laan A C, Vasile E, et al. Correlation between cytidine deaminase genotype and gemcitabine deamination in blood samples. Nucleosides Nucleotides Nucleic Acids. 27: 720-5, 2008.

9. Xiang T X, Niemi R, Bummer P, et al. Epimer interconversion, isomerization, and hydrolysis of tetrahydrouridine: implications for cytidine deaminase inhibition. J Pharm Sci. 92: 2027-39, 2003.

10. Kirstein M N, Hassan I, Guire D E, et al. High-performance liquid chromatographic method for the determination of gemcitabine and 2′,2′-difluorodeoxyuridine in plasma and tissue culture media. J Chromatogr B Analyt Technol Biomed Life Sci. 835: 136-42, 2006.

11. Claret L, Iliadis A, Macheras P A stochastic model describes the heterogeneous pharmacokinetics of cyclosporin.J Pharmacokinet Pharmacodyn. 28: 445-63, 2001.

12. Mercier C, Raynal C, Dahan L, et al. Toxic death case in a patient undergoing gemcitabine-based chemotherapy in relation with cytidine deaminase downregulation. Pharmacogenet Genomics. 17: 841-4, 2007.

13. Danesi R, Altavilla G, Giovannetti E, Rosell R. Pharmacogenomics of gemcitabine in non-small-cell lung cancer and other solid tumors. Pharmacogenomics. 10: 69-80, 2009.

14. Sugiyama E, Kaniwa N, Kim S R, et al. Pharmacokinetics of gemcitabine in Japanese cancer patients: the impact of a cytidine deaminase polymorphism. J Clin Oncol. 25: 32-42, 2007.

15. Ueno H, Kaniwa N, Okusaka T, et al. Homozygous CDA*3 is a major cause of life-threatening toxicities in gemcitabine-treated Japanese cancer patients. Br J Cancer. 100: 870-3, 2009.

16. Mercier C, Evrard A, Ciccolini J. Genotype-based methods for anticipating gemcitabine-related severe toxicities may lead to false-negative results. J Clin Oncol. 25: 4855-6, 2007.

17. Matsubara J, Ono M, Negishi A, et al. Identification of a Predictive Biomarker for Hematologic Toxicities of Gemcitabine. J Clin Oncol. 27: 2261-8, 2009.

18. Glezerman I, Kris M G, Miller V, et al. Gemcitabine nephrotoxicity and hemolytic uremic syndrome: report of 29 cases from a single institution. Clin Nephrol. 71: 130-9, 2009.

19. Matsubara J, Ono M, Okusaka T, et al. Predictive biomarker for hematological toxicities of gemcitabine identified by proteomics. Proc Am Ass Can Res. 2009 (abstract #695).

20. Mercier C, Dupuis C, Blesius A, et al. Early severe toxicities after capecitabine intake: possible implication of a cytidine deaminase extensive metabolizer profile. Cancer Chemother Pharmacol. 63: 1177-80, 2009.

21. Felici A, Di Segni S, Milella M, et al. Pharmacokinetics of gemcitabine at fixed-dose rate infusion in patients with normal and impaired hepatic function. Clin Pharmacokinet. 48: 131-41, 2009.

22. Abbruzzese J L, Grunewald R, Weeks E A, et al. A phase I clinical, plasma, and cellular pharmacology study of gemcitabine. J Clin Oncol. 9: 491-8, 1991.

23. Navid F, Willert J R, McCarville M B, et al. Combination of gemcitabine and docetaxel in the treatment of children and young adults with refractory bone sarcoma. Cancer. 113: 419-25, 2008.

24. Cole P D, Schwartz C L, Drachtman R A, et al. Phase II study of weekly gemcitabine and vinorelbine for children with recurrent or refractory Hodgkin's disease: a children's oncology group report. J Clin Oncol. 27: 1456-61, 2009.

1-14. (canceled)

15. A method of treating a patient having cancer treatable with at least one chemotherapeutic agent, said chemotherapeutic agent being eliminated by cytidine deaminase (CDA) in the liver, said method comprising determining the CDA activity in a biological sample of the patient and administering at least one chemotherapeutic agent which is eliminated in the liver by CDA to a patient having a cancer treatable with a chemotherapeutic agent eliminated by cytidine deaminase (CDA) in the liver, wherein:
a) the chemotherapeutic agent is administered as a monotherapy to a patient having a CDA activity between 1.1 CDA activity unit per milligram of serum sample total protein (1.1 U/mg) and 6 CDA activity unit per milligram of serum sample total protein (6 U/mg); or
b) the chemotherapeutic agent is administered as a component of a polychemotherapy to a patient having a CDA activity between 1.4 CDA activity unit per milligram of serum sample total protein (1.4 U/mg) and 6 CDA activity unit per milligram of serum sample total protein (6 U/mg).

16. The method of claim 15, wherein the chemotherapeutic agent is selected from gemcitabine, Ara-Cytidine (Ara-C), CNDAC, decitabine, 5-aza-cytidine, clofarabine, nelarabine, troxacitabine, 2′-deoxy-2′-methylidenecytidine (DMDC) or tezacitabine.

17. The method of claim 16, wherein the chemotherapeutic agent is gemcitabine.

18. The method of claim 15, wherein the chemotherapeutic agent is administered as a component of a polychemotherapy, together with an agent selected from an alkylating agent, an antimetabolite, an antimitotic agent, a monoclonal antibody or a tyrosine-kinase inhibitor.

19. The method of claim 18, wherein the alkylating agent is selected from oxaliplatin, cisplatin or carboplatin; the antimetabolite is selected from capecitabine or 5-fluorouracil; the monoclonal antibody is herceptin, the antimitotic agent is selected from docetaxel or navelbin; and the tyrosine-kinase inhibitor is selected from erlotinib or sorafenib.

20. The method of claim 15, wherein the CDA activity is determined by spectrophotometry.

21. The method of claim 20, wherein the CDA activity is determined by visible spectrophotometry.

22. The method of claim 15, wherein the CDA activity is determined by measuring the amount of ammonium released through conversion of cytidine into uridine, by spectrophotometry.

23. The method of claim 22, wherein CDA activity is measured by an assay comprising:
a) incubating the biological sample with cytidine;
b) setting up a calibration curve of ammonium to be incubated similarly with the sample;
c) precipitating proteins so as to stop the reaction;
d) centrifuging and recovering the upper layer;
e) incubating the recovered upper layer of step d) with a mixture of phenol and sodium hypochlorite and recovering the upper layer comprising the ammonium;
f) detecting ammonium in the recovered upper layer of step e) with a spectrophotometer; and
g) calculating the CDA activity, in regard to the signal/activity relationship generated by the calibration curve and the amount of proteins in the sample.

24. A kit for determining the ability of a patient with cancer to be treated by at least one chemotherapeutic agent the liver elimination of which involves cytidine deaminase (CDA), which kit comprises:
i) a container comprising cytidine;
ii) a container comprising ammonium; and
iii) a leaflet which describes a method for determining the cytidine deaminase (CDA) activity in a biological sample, by measuring the amount of ammonium released through conversion of cytidine into uridine by spectrophotometry, wherein the leaflet indicates that:
a CDA activity between 1.1 and 6 U/mg, in a serum sample, is indicative of the ability of the patient to be treated by the chemotherapeutic agent the liver elimination of which involves CDA, when said agent is administered in the context of a monochemotherapy; or
a CDA activity between 1.4 and 6 U/mg, in a serum sample, is indicative of the ability of the patient to be treated by the chemotherapeutic agent the liver elimination of which involves CDA, when said agent is administered in the context of a polychemotherapy.

25. The kit of claim 24, wherein the chemotherapeutic agent the liver elimination of which involves CDA is selected from the group consisting of gemcitabine, Ara-Cytidine (Ara-C), CNDAC, decitabine, 5-aza-cytidine, clofarabine, nelarabine, troxacitabine, 2′-deoxy-2′-methylidenecytidine (DMDC) and tezacitabine.

26. The kit of claim 24, said kit further comprising:
i) a container comprising sodium and phosphate buffers;
ii) a container comprising phenol and nitroprusside;
iii) a container comprising tungstate;
iv) a container comprising hypochlorite; and
v) a container comprising sulphuric acid.

27. A kit for determining the ability of a patient with cancer to respond to a monochemotherapy or to a polychemotherapy involving the administration of at least one chemotherapeutic agent the liver elimination of which involves cytidine deaminase (CDA), which kit comprises:
i) a container comprising cytidine;
ii) a container comprising ammonium; and
iii) a leaflet which describes a method for determining the cytidine deaminase (CDA) activity in a biological sample, by measuring the amount of ammonium released through conversion of cytidine into uridine by spectrophotometry, wherein the leaflet indicates that a CDA activity below 6 U/mg, in a serum sample, is indicative of the ability of the patient to respond to the chemotherapeutic agent whereas a CDA activity above 6 U/mg is indicative of the inability of the patient to respond to the chemotherapy.

28. The kit of claim 27, wherein the chemotherapeutic agent the liver elimination of which involves CDA is selected from the group consisting of gemcitabine, Ara-Cytidine (Ara-C), CNDAC, decitabine, 5-aza-cytidine, clofarabine, nelarabine, troxacitabine, 2′-deoxy-2′-methylidenecytidine (DMDC) and tezacitabine.

29. The kit of claim 28, said kit further comprising:
i) a container comprising sodium and phosphate buffers;
ii) a container comprising phenol and nitroprusside;
iii) a container comprising tungstate;
iv) a container comprising hypochlorite; and
v) a container comprising sulphuric acid.
